(19) United States
(12) Patent Application Publication
(10) Pub. No.: US 2020/0113795 A1
(43) Pub. Date: Apr. 16, 2020

(54) COSMETIC COMPOSITION AND PREPARATION METHOD THEREFOR

(71) Applicant: AMOREPACIFIC CORPORATION, Seoul (KR)

(72) Inventors: Seung-Hwan YI, Yongin-si (KR); So Mi LEE, Yongin-si (KR)

(73) Assignee: AMOREPACIFIC CORPORATION, Seoul (KR)

(21) Appl. No.: 16/627,025

(22) PCT Filed: Jun. 29, 2018
(86) PCT No.: PCT/KR2018/007391

§ 371 Date: Dec. 27, 2019

(30) Foreign Application Priority Data
Jun. 30, 2017 (KR) 10-2017-0083089
Jun. 26, 2018 (KR) 10-2018-0073391

(51) Int. Cl. A61K 8/29; A61K 8/58; A61K 8/368; A61K 8/44; A61K 8/365; A61K 8/64; A61Q 1/00; A61K 8/34; A61Q 19/00; A61K 8/60; A61K 8/49

(52) U.S. Cl. CPC A61K 8/29; A61K 8/58; A61K 8/368; A61K 8/44; A61K 8/365; A61K 8/4926; A61Q 1/00; A61K 8/347; A61Q 19/00; A61K 8/606; A61K 8/64

(57) ABSTRACT

One aspect of the present disclosure relates to a cosmetic composition including an inorganic powder; at least one stabilizer selected from the group consisting of a metal dispersing agent, an anionic surfactant and a pH adjusting agent; and a functional active component. According to the cosmetic composition according to one aspect of the present disclosure, while maintaining the dispersion stability and even distribution in the skin, it is possible to prevent the functional active components from adsorbing on the inorganic powder.

### TECHNICAL FIELD

This application claims the benefits of Korean Patent Application No. 10-2017-0083089 filed on Jun. 30, 2017 and Korean Application No. 10-2018-0073391 filed on Jun. 26, 2018 with the Korean Intellectual Property Office, the entire contents of which are hereby incorporated by reference.

One aspect of the present disclosure is relates to a cosmetic composition, and more particularly to a cosmetic composition stably showing an excellent stability of the active component and an excellent make-up effect.

### BACKGROUND ART

If particulates of submicron size among the inorganic powder such as titanium dioxide or zinc oxide are contained in the cosmetic composition, an ultraviolet blocking agent that is less irritating than an organic ultraviolet blocking agent can be provided while protecting a wide range of ultraviolet spectroscopy (UV-A,B), and the dispersion stability in the composition can be improved, and also it has the advantage of having an even distribution when applied to the skin, and thus it is used in cosmetic compositions. On the other hand, materials having physical properties of reflecting and dispersing ultraviolet rays may be titanium dioxide, iron oxide, magnesium oxide, and the like, in addition to titanium dioxide and zinc oxide.

In addition, various surface treatment technologies have been developed to modify the surface of the particulates to further secure the dispersion stability and even distribution in the skin.

However, in the case of the inorganic powder, it has a large specific surface area (m²/g) and surface charge due to atomization. In particular, if surface modification is insufficient, there was a problem that the functional active components such as adenosine, niacinamide, phenoxy ethanol, ethylhexylmethoxy cinnamate, etc. are adsorbed on the inorganic powder, resulting in a decrease in the content of the functional active components contained in the cosmetic composition.

Therefore, there is a need for the development of a cosmetic composition that can prevent the functional active components from adsorbing on the inorganic powder, while maintaining the dispersion stability and even distribution in the skin as described above.

[Patent Document]

Korean Laid-open Patent Publication No. 10-2000-0026082, “COMPLEX PIGMENT FOR COSMETICS AND MANUFACTURING PROCESS”

### DISCLOSURE

### Technical Problem

In order to solve the above problems, applicants of the present invention used a metal dispersing agent, an anionic surfactant or a pH adjusting agent, and the like along with an inorganic powder in a cosmetic composition, and as a result, confirmed that it is possible to maintain the dispersion stability of functional active components and their even distribution in the skin, while preventing the functional active components from adsorbing on the inorganic powder.

Therefore, it is an object of one aspect of the present disclosure to provide a cosmetic composition that can prevent the functional active components from adsorbing on the inorganic powder, while maintaining the dispersion stability and even distribution in the skin.

### Technical Solution

In order to solve the above problems, one aspect of the present disclosure provides a cosmetic composition comprising an inorganic powder; at least one stabilizer selected from the group consisting of a metal dispersing agent, an anionic surfactant and a pH adjusting agent; and a functional active component.

In that case, the metal dispersing agent may be at least one selected from the group consisting of a gluconate-based metal salt, a stearate-based metal salt, an aspartate-based metal salt, and a cetyl phosphate-based metal salt In that case, the metal dispersing agent may be at least one selected from the group consisting of copper (Cu) gluconate, calcium (Ca) gluconate, zinc (Zn) gluconate, calcium (Ca) stearate, magnesium (Mg) stearate, zinc (Zn) stearate, aluminum (Al) stearate, magnesium (Mg) aspartate and potassium (K) cetyl phosphate.

In that case, the anionic surfactant may be at least one selected from the group consisting of glyceryl stearate citrate, sodium surfactin, polyoxyethylene glycerylisostearate, PEG-20 glyceryl triisostearate, polyethylene glycol monoisostearate, PEG-100 stearate, PEG-40 stearate, sorbitan stearate, glyceryl laurate, glyceryl stearate and hexahydroxy stearate.

In that case, the pH adjusting agent may be at least one selected from the group consisting of L-glutamic acid, lactic acid, citric acid, salicylic acid, myristic acid, oleic acid and lauric acid.

In that case, the inorganic powder may be at least one selected from the group consisting of titania (TiO₂), zinc oxide (ZnO), iron oxide (Fe₂O₃, FeO, Fe₃O₄), magnesium oxide (MgO), manganese oxide (MnO₂) and cerium oxide (CeO).

In that case, the functional active component may be at least one selected from the group consisting of adenosine, niacinamide, phenoxyethanol and ethylhexylmethoxy cinnamate.

In that case, the inorganic powder and the stabilizer may be contained in a ratio of 100:1 to 10:1.

In that case, the inorganic powder may be contained in an amount of 1 to 10 wt. % based on the total weight of the composition.

In that case, the stabilizer may be contained in an amount of 0.05 to 1.0 wt. % based on the total weight of the composition.

In that case, some of the stabilizers may be combined with the OH group on the surface of the inorganic powder or combined with a condensation reaction.

In that case, the cosmetic composition may be used in a cosmetic composition for skin care or a cosmetic composition for make-up.

### Advantageous Effects

According to the present invention, by using a metal dispersing agent, an anionic surfactant, a pH adjusting agent or the like along with an inorganic powder, it is possible to maintain the dispersion stability of the functional active components and their even distribution in the skin, while preventing the functional active components from adsorbing on the inorganic powder.

Therefore, it is possible to prepare a cosmetic composition which can stably contain functional active components in the composition.

As such, the product having the dispersion stability and even distribution in the skin may be a product such as a cosmetic composition for skin care or a cosmetic composition for make-up, and it is possible to improve preference for these products.

### BEST MODE

One aspect of the present disclosure proposes a cosmetic composition that can prevent the adsorption of functional active components onto inorganic powders, while maintaining the dispersion stability and even distribution in the skin, wherein the cosmetic composition comprises an inorganic powder; at least one stabilizer selected from the group consisting of a metal dispersing agent, a anionic surfactant and a pH adjusting agent; and a functional active component.

In order to increase the dispersion stability in the cosmetic composition, if the inorganic powder particulates such as titanium dioxide or zinc oxide are used, there was a problem that the functional active components such as adenosine, niacinamide, phenoxy ethanol, ethylhexylmethoxy cinnamate, etc. are adsorbed on the inorganic powder due to the large specific surface area (m²/g) and the presence of surface charges depending on the atomization of the inorganic powder, resulting in a reduction in the content of the functional active components contained in the cosmetic composition.

One aspect of the present disclosure relates to a technique for implementing a cosmetic composition that can prevent the adsorption of the functional active components on the inorganic powder, while maintaining the dispersion stability and even distribution in the skin, by using a stabilizer such as a metal dispersing agent, an anionic surfactant, and a pH adjusting agent together with the inorganic powder. This technique has a ripple effect to the customer that it is possible to stably maintain the content of the functional active components contained in the cosmetic composition, which is a feature of this product.

The inorganic powder contained in the cosmetic composition of one aspect of the present disclosure is not particularly limited as long as it is an inorganic powder having a size (particle diameter) of less than a micron (μm), but preferably may be at least one selected from the group consisting of titania (TiO₂), zinc oxide (ZnO), iron oxide (Fe₂O₃, FeO, Fe₃O₄), magnesium oxide (MgO), manganese oxide (MnO₂), cerium oxide (CeO) and the like.

The inorganic powder may be contained in an amount of 0.1 to 25 wt. %, preferably 0.5 to 15 wt. %, more preferably 1 to 10 wt. % based on the total weight of the composition. If the inorganic powder is contained in an amount of less than 0.1 wt. %, the ultraviolet blocking effect is insignificant. If the inorganic powder is used in more than 25 wt. %, there is a disadvantage that it is not cosmetically appropriate. The expression “it is not cosmetically appropriate” means that due to smeary feeling, there is no refreshing feeling and thus an unpleasant feeling is caused and that due to the white turbidity phenomenon on the skin, the skin looks quite pale and greasy which is bad in appearance. In addition, the excess physical blocking agent dispersed in the nano-size can penetrate into the skin, and thus is likely to cause problems in terms of safety and may cause skin irritation and skin damage due to the photoactivity resulting from free radicals, and has a disadvantage in terms of functionality that the ultraviolet blocking ability is degraded over time due to an increase in particle size by the secondary aggregation of particles. Therefore, the compounding limit of the physical blocking agent under the Korean Cosmetics Act is limited to 25 wt. % or less.

The stabilizer contained in the cosmetic composition of one aspect of the present disclosure may be at least one selected from the group consisting of a metal dispersing agent, an anionic surfactant and a pH adjusting agent. The stabilizer may be contained in an amount of 0.05 to 20.0 wt. %, preferably 0.1 to 5 wt. %, based on the total weight of the composition. If the stabilizer is contained in an amount of less than 0.05 wt. %, since it is not possible to prevent the functional active components from adsorbing on the inorganic powder, there is a problem in terms of stability of the functional active component. If the stabilizer is contained in an amount of more than 20 wt. %, It is not preferable because of problems such as formulation stability and skin irritation.

The metal dispersing agent may be at least one selected from the group consisting of a gluconate-based metal salt, stearate-based metal salt, an aspartate-based metal salt, a cetyl phosphate-based metal salt and the like, and more specifically may be at least one selected from the group consisting of copper (Cu) gluconate, calcium (Ca) gluconate, zinc (Zn) gluconate, calcium (Ca) stearate, magnesium (Mg) stearate, zinc (Zn) stearate, aluminum (Al) stearate, magnesium (Mg) aspartate, potassium (K) cetyl phosphate and the like.

The anionic surfactant may be at least one selected from the group consisting of glyceryl stearate citrate, sodium surfactin, polyoxyethylene glycerylisostearate, PEG-20 glyceryl triisostearate, polyethylene glycol monoisostearate, PEG-100 stearate, PEG-40 stearate, sorbitan stearate, glyceryl laurate, glyceryl stearate, hexahydroxy stearate and the like.

The pH adjusting agent may be at least one selected from the group consisting of L-glutamic acid, lactic acid, citric acid, salicylic acid, myristic acid, oleic acid, lauric acid and the like. All or part of the stabilizer can be bound to OH groups on the surface of the inorganic powder, or bound by a condensation reaction, or an ionic bond (in the case of a metal dispersing agent) to prevent the functional active components from adsorbing on the inorganic powder. Through this, it is possible to increase the content of the functional active components substantially present in the cosmetic composition. Therefore, the functional active components adsorbed on the inorganic powder over time can be minimized, and even if the cosmetic composition is used for a long time, the functional active components can be stably contained.

To this end, the inorganic powder and stabilizer may be contained in an ratio of 100:1 to 10:1 (inorganic powder:stabilizer). If the content ratio of the inorganic powder and the stabilizer exceeds 100:1, there is a problem that the stabilizer does not sufficiently react to the inorganic powder, and thus does not prevent the adsorption of the functional active components. If the ratio of the inorganic powder and stabilizer is less than 10:1, there are problems that the pH adjusting agent causes the pH to be too low, and that the anionic surfactant or the metal dispersing agent causes the skin irritation to occur, which are not desirable.

The functional active component contained in the cosmetic composition of one aspect of the present disclosure may be at least one selected from the group consisting of adenosine, niacinamide, phenoxyethanol, ethylhexylmethoxycinnamate, and the like.

The functional active component may be used in various compositions and contents according to a user's intention, and preferably may legally contain in Korea up to 0.04% of adenosine as a content of the raw material notified for wrinkle improvement functionality in a functional cosmetic product, 2.00% of niacinamide as a content of the raw material notified for whitening functionality in a functional cosmetic product, 2.00% of albumin as a content of the raw material notified for whitening functionality in a functional cosmetic product, 7.50% of a Food and Drug Administration (FDA) limit concentration of ethylhexylmethoxycinnamate as a content of the raw material notified for ultraviolet blocking functionality in a functional cosmetic product, and 1% of phenoxyethanol, based on the total weight of the composition.

In addition to the above components, the cosmetic composition of one aspect of the present disclosure may further contain components used in a conventional cosmetic composition. There are no particular limitations to the additional components, but the cosmetic composition may further contain Broussonetia kazinokii extract, oil-soluble licorice extract, retinyl palmitate, polyethoxylated retinamide, ascorbyl glucoside, magnesium ascorbyl phosphate, and the like.

The cosmetic composition of one aspect of the present disclosure can be used in the cosmetic product that can maintain the dispersion stability and even distribution in the skin, and the cosmetic composition comprises both a cosmetic composition for skin care or a cosmetic composition for make-up, and specifically may be formulated as make-up primer, make-up base, foundation, powder, twin cake, lipstick, lipgloss, eye shadow, eyebrow, concealer, lip liner, blusher, ultraviolet blocking agent, lotion, cream or essence, more specifically make-up primer, make-up base, liquid-phase or solid-phase foundation, powder, twin cake, lipstick, lipgloss, eye shadow, eyebrow, concealer or blusher, but is not limited thereto.

In addition, one aspect of the present disclosure provides a method for preparing the cosmetic composition comprising the steps of a) mixing the stabilizer with the inorganic powder, and then reacting in an aqueous phase or an oil phase; and b) mixing the functional active component with the obtained reactant.

In the method for preparing the cosmetic composition, the description for the inorganic powder, the stabilizer and the functional active component is the same as described in the cosmetic composition of one aspect of the present disclosure.

Hereinafter, the preparing method of the cosmetic composition of one aspect of the present disclosure will be described in more detail with reference to examples of the present invention. It is to be understood that the present invention is not limited to these examples.

### EXAMPLE

The oil phase component and the aqueous phase component, the inorganic powder, the stabilizer and the functional active component as shown in Table 1 were mixed to prepare a cosmetic composition.

TABLE 1

| | | Content |
|---|---|---|
| Component | Name of raw material | (wt. %) |
| Oil phase | Cyclopentasiloxae (DC556, Dowcoring | 12% |
| component | company) | |
| | PEG 10 DIMETHICONE (Gransufr87, | 1% |
| | GRANT company) | |
| | Cyclopentasiloxane & | 15% |
| | Trimethylsiloxysilicate & C24-28 alkyl | |
| | dimethicone (Granresin MQC-TA, GRANT | |
| | company) | |
| | Dimethicone & PEG/PPG-18/18 Dimethicone | 2% |
| | (GRANSUFR 50C-HM, Grant company) | |
| | Disteardimonium hectorite & PROPYLENE | 0.3% |
| | CARBONATE & C9-13 ISOPARAFFIN | |
| | (Bentone Gel ® 10-ST-V, | |
| | Elementis company) | |
| | TRIBEHENIN(Lipovol ® GTB, Vantage | 0.5% |
| | Specialty Ingredients) | |
| | LAURETH-7 (PEL-ALC ™ LA-7 90, | 0.5% |
| | ELE company) | |
| Aqueous | DEIONIZED WATER | Remainder |
| phase | BUTYLENE GLYCOL (1,3-Butylene glycol, | 5% |
| component | Daiichi fine chem company) | |
| | PHENOXYETHANOL | 0.3% |
| | (PHENOXYETHANOL, Galaxy Surfactantsk) | |
| | DISODIUM EDTA (Edeta BD, BASF | 0.05% |
| | company) | |
| | Sodium chloride | 0.5% |
| | PERFUME | 0.3% |
| Inorganic | Particulate titanium oxide | |
| powder | | |
| Stabilizer | Example | |
| Functional | Example | |
| active | | |
| component | | |

As the stabilizer and the functional active component, the stabilizers and the functional active components shown in Tables 2 to 3 were used. Specific preparing method is as follows.

### Examples 1 to 16 and Comparative Example 1

The stabilizers and the functional active components shown in Table 2 were used.

1) Each of 5 wt. % of the oil phase component and the particulate titanium oxide of Table 1 were mixed, and 0.4 wt. % of the stabilizer of Table 2 was mixed, followed by mixing and dispersing at 65 for 5 minutes.

2) Thereafter, the aqueous phase component of Table 1 and the functional active component of Table 2 were all mixed in an amount of 0.04 wt. % respectively.

3) The obtained mixture 2) was slowly added to the obtained mixture 1) and mixed, followed by complete degassing to prepare a water in oil type make-up cosmetic composition. The compositions of Examples 1 to 16 were prepared as water in oil type cosmetic compositions. In addition, the composition of Comparative Example 1 was prepared in the same manner except that no stabilizer was used.

TABLE 2

| | | Functional |
|---|---|---|
| | Stabilizer | active component |
| Example 1-1 | Copper Gluconate | Adenosine |
| Example 1-2 | | Niacinamide |
| Example 1-3 | | Phenoxyethanol |
| Example 2-1 | Calcium Gluconate | Adenosine |
| Example 2-2 | | Niacinamide |
| Example 2-3 | | Phenoxyethanol |
| Example 3-1 | Calcium-stearate | Adenosine |
| Example 3-2 | | Niacinamide |
| Example 3-3 | | Phenoxyethanol |
| Example 4-1 | Aluminum stearate | Adenosine |
| Example 4-2 | | Niacinamide |
| Example 4-3 | | Phenoxyethanol |
| Example 5-1 | Magnesium Aspartate | Adenosine |
| Example 5-2 | | Niacinamide |
| Example 5-3 | | Phenoxyethanol |
| Example 6-1 | GLYCERYL STEARATE | Adenosine |
| Example 6-2 | CITRATE | Niacinamide |
| Example 6-3 | | Phenoxyethanol |
| Example 7-1 | POLYOXYETHYLENE | Adenosine |
| Example 7-2 | GLYCERYL | Niacinamide |
| Example 7-3 | ISOSTEARATE | Phenoxyethanol |
| Example 8-1 | SODIUM SURFACTIN | Adenosine |
| Example 8-2 | | Niacinamide |
| Example 8-3 | | Phenoxyethanol |
| Example 9-1 | PEG-20 Glyceryl | Adenosine |
| Example 9-2 | Triisostearate | Niacinamide |
| Example 9-3 | | Phenoxyethanol |
| Example 10-1 | POLYETHYLENE GLYCOL | Adenosine |
| Example 10-2 | MONOISOSTEARATE | Niacinamide |
| Example 10-3 | | Phenoxyethanol |
| Example 11-1 | GLYCERYL STEARATE | Adenosine |
| Example 11-2 | SE | Niacinamide |
| Example 11-3 | | Phenoxyethanol |
| Example 12-1 | Sorbitan Stearate | Adenosine |
| Example 12-2 | | Niacinamide |
| Example 12-3 | | Phenoxyethanol |
| Example 13-1 | Glyceryl laurate | Adenosine |
| Example 13-2 | | Niacinamide |
| Example 13-3 | | Phenoxyethanol |
| Example 14-1 | Hexahydroxystearate | Adenosine |
| Example 14-2 | | Niacinamide |
| Example 14-3 | | Phenoxyethanol |
| Example 15-1 | L-glutamic acid | Adenosine |
| Example 15-2 | | Niacinamide |
| Example 15-3 | | Phenoxyethanol |
| Example 16-1 | LACTIC ACID | Adenosine |
| Example 16-2 | | Niacinamide |
| Example 16-3 | | Phenoxyethanol |
| Comparative | — | Adenosine |
| Example 1-1 | | |
| Comparative | | Niacinamide |
| Example 1-2 | | |
| Comparative | | Phenoxyethanol |
| Example 1-3 | | |

### Examples 17 to 30 and Comparative Example 2

The stabilizers and functional active components shown in Table 3 were used.

1) Each of 5 wt. % of the oil phase component and the particulate titanium oxide of Table 1 were mixed, and 0.4 wt. % of the stabilizer of Table 3 was mixed, followed by mixing and dispersing at 90° C. for 15 minutes.

2) Thereafter, the aqueous phase component of Table 1 and the functional active component of Table 3 were all mixed in an amount of 0.04 wt. % respectively.

3) The obtained mixture 2) was slowly added to the obtained mixture 1) and mixed, followed by complete degassing to prepare a water in oil type make-up cosmetic composition. The compositions of Examples 17 to 29 were prepared as water in oil type cosmetic compositions. In addition, the composition of Comparative Example 2 was prepared in the same manner except that no stabilizer was used.

TABLE 3

| | | Functional active |
|---|---|---|
| | Stabilizer | component |
| Example 17-1 | Copper Gluconate | Adenosine |
| Example 17-2 | | Niacinamide |
| Example 18-1 | Calcium Gluconate | Adenosine |
| Example 18-2 | | Niacinamide |
| Example 19-1 | Calcium-stearate | Adenosine |
| Example 19-2 | | Niacinamide |
| Example 20-1 | Aluminum stearate | Adenosine |
| Example 20-2 | | Niacinamide |
| Example 21-1 | Magnesium Aspartate | Adenosine |
| Example 21-2 | | Niacinamide |
| Example 22-1 | GLYCERYL STEARATE | Adenosine |
| Example 22-2 | CITRATE | Niacinamide |
| Example 23-1 | POLYOXYETHYLENE | Adenosine |
| Example 23-2 | GLYCERYL ISOSTEARATE | Niacinamide |
| Example 24-1 | SODIUM SURFACTIN | Adenosine |
| Example 24-2 | | Niacinamide |
| Example 25-1 | PEG-20 Glyceryl | Adenosine |
| Example 25-2 | Triisostearate | Niacinamide |
| Example 26-1 | POLYETHYLENE GLYCOL | Adenosine |
| Example 26-2 | MONOISOSTEARATE | Niacinamide |
| Example 27-1 | GLYCERYL STEARATE SE | Adenosine |
| Example 27-2 | | Niacinamide |
| Example 28-1 | Sorbitan Stearate | Adenosine |
| Example 28-2 | | Niacinamide |
| Example 29-1 | Glyceryl laurate | Adenosine |
| Example 29-2 | | Niacinamide |
| Comparative | — | Adenosine |
| Example 1-1 | | |
| Comparative | | Niacinamide |
| Example 1-2 | | |

### Experimental Example

For the cosmetic compositions of Comparative Examples and Examples, HLPC analysis was performed on compositions immediately after preparation and compositions after 24 weeks, and is described in Tables 4 to 5 below. The conditions are as follows.
Column: CAPCELL PAK C18(SHISEIDO)
Detector (wavelength): UV absorbance detector (203 nm)
Injection volume, flow rate: 20 uL, 1.0 mL/min
Mobile phase: Gradient conditions for HPLC (A: D.I WATER, B: 50% BeCN)

TABLE 4

| | | Functional active component | | |
|---|---|---|---|---|
| | Stabilizer | 1: | 2: | 3: |
| | component | Adenosine | Niacinamide | Phenoxyethanol |
| Example 1 | Copper | 97.7% | 98.8% | 99.4% |
| | Gluconate | | | |
| Example 2 | Calcium | 97.3% | 98.4% | 99.5% |
| | Gluconate | | | |
| Example 3 | Calcium- | 99.3% | 100% | 99.9% |
| | stearate | | | |
| Example 4 | Aluminum | 97.2% | 98.5% | 98.0% |
| | stearate | | | |
| Example 5 | Magnesium | 98.3% | 98.9% | 98.2% |
| | Aspartate | | | |
| Example 6 | GLYCERYL | 99.8% | 100% | 99.9% |
| | STEARATE | | | |
| | CITRATE | | | |
| Example 7 | POLYOXYETHYLENE | 99.5% | 100% | 98.9% |
| | GLYCERYL | | | |
| | ISOSTEARATE | | | |
| Example 8 | SODIUM | 96.2% | 99.2% | 99.1% |
| | SURFACTIN | | | |
| Example 9 | PEG-20 Glyceryl | 97.4% | 99.4% | 99.5% |
| | Triisostearate | | | |
| Example 10 | POLYETHYLENE | 98.8% | 99.4% | 99.4% |
| | GLYCOL | | | |
| | MONOISOSTEARATE | | | |
| Example 11 | GLYCERYL | 99.1% | 100% | 98.9% |
| | STEARATE SE | | | |
| Example 12 | Sorbitan | 96.9% | 98.8% | 98.8% |
| | Stearate | | | |
| Example 13 | glyceryl | 97.5% | 99.1% | 99.6% |
| | laurate | | | |
| Example 14 | Hexahydroxystearate | 94.5% | 98.9% | 97.7% |
| Example 15 | L-glutamic acid | 93.0% | 97.4% | 98.6% |
| Example 16 | LACTIC ACID | 93.7% | 96.8% | 97.8% |
| Comparative | — | 69.392% | 87.7% | 96.2% |
| Example 1 | | | | |

TABLE 5

| | Stabilizer | Functional active component | |
|---|---|---|---|
| | component | 1: Adenosine | 2: Niacinamide |
| Example 17 | Copper Gluconate | 91% | 92.5% |
| Example 18 | Calcium | 89.4% | 93.4% |
| | Gluconate | | |
| Example 19 | Calcium-stearate | 92.3% | 94.3% |
| Example 20 | Aluminum | 93.1% | 94.2% |
| | stearate | | |
| Example 21 | Magnesium | 90.4% | 93.2% |
| | Aspartate | | |
| Example 22 | GLYCERYL | 95.1% | 92.7% |
| | STEARATE CITRATE | | |
| Example 23 | POLYOXYETHYLENE | 94.5% | 94.4% |
| | GLYCERYL | | |
| | ISOSTEARATE | | |
| Example 24 | SODIUM SURFACTIN | 93.7% | 91.8% |
| Example 25 | PEG-20 Glyceryl | 88.5% | 92.3% |
| | Triisostearate | | |
| Example 26 | POLYETHYLENE | 90.1% | 93.1% |
| | GLYCOL | | |
| | MONOISOSTEARATE | | |
| Example 27 | GLYCERYL | 92.0% | 93.1% |
| | STEARATE SE | | |
| Example 28 | Sorbitan | 92.9% | 92.0% |
| | Stearate | | |
| Example 29 | glyceryl laurate | 90.3% | 91.2% |
| Comparative | — | 69.92% | 87.7% |
| Example 2 | | | |

As shown in Tables 4 and 5, it can be seen that in the case of the cosmetic composition of Examples 1 to 29 with the stabilizer, even after 24 weeks, the content of the functional active components such as adenosine, niacinamide, and phenoxyethanol is not significantly decreased. On the other hand, it can be seen that in the case of the cosmetic compositions of Comparative Examples 1 to 2 without the stabilizer, the content of the functional active components over the course of 24 weeks.

Therefore, it can be seen that if the cosmetic composition of one aspect of the present disclosure is used, the functional active components are not adsorbed on the inorganic powder even after a long time, so that the functional active components can be stably contained.

1. A cosmetic composition comprising an inorganic powder; at least one stabilizer selected from the group consisting of a metal dispersing agent, an anionic surfactant and a pH adjusting agent; and a functional active component.

2. The cosmetic composition according to claim 1, wherein the metal dispersing agent is at least one selected from the group consisting of a gluconate-based metal salt, a stearate-based metal salt, an aspartate-based metal salt, and a cetyl phosphate-based metal salt.

3. The cosmetic composition according to claim 1, wherein the metal dispersing agent is at least one selected from the group consisting of copper (Cu) gluconate, calcium (Ca) gluconate, zinc (Zn) gluconate, calcium (Ca) stearate, magnesium (Mg) stearate, zinc (Zn) stearate, aluminum (Al) stearate, magnesium (Mg) aspartate, and potassium (K) cetyl phosphate.

4. The cosmetic composition according to claim 1, wherein the anionic surfactant is at least one selected from the group consisting of glyceryl stearate citrate, sodium surfactin, polyoxyethylene glycerylisostearate, PEG-20 glyceryl triisostearate, polyethylene glycol monoisostearate, PEG-100 stearate, PEG-40 stearate, sorbitan stearate, glyceryl laurate, glyceryl stearate, and hexahydroxy stearate.

5. The cosmetic composition according to claim 1, wherein the pH adjusting agent is at least one selected from the group consisting of L-glutamic acid, lactic acid, citric acid, salicylic acid, myristic acid, oleic acid, and lauric acid.

6. The cosmetic composition according to claim 1, wherein the inorganic powder is at least one selected from the group consisting of titania (TiO₂), zinc oxide (ZnO), iron oxide (Fe₂O₃, FeO, Fe₃O₄), magnesium oxide (MgO), manganese oxide (MnO₂), and cerium oxide (CeO).

7. The cosmetic composition according to claim 1, wherein the functional active component is at least one selected from the group consisting of adenosine, niacinamide, phenoxyethanol, and ethylhexylmethoxy cinnamate.

8. The cosmetic composition according to claim 1, wherein the inorganic powder and stabilizer are contained in a ratio of 100:1 to 10:1.

9. The cosmetic composition according to claim 1, wherein the inorganic powder is contained in an amount of 1 to 10 wt. % based on the total weight of the composition.

10. The cosmetic composition according to claim 1, wherein the stabilizer is contained in an amount of 0.05 to 1.0 wt. % based on the total weight of the composition.

11. The cosmetic composition according to claim 1, wherein some of the stabilizers are bound to OH groups on the surface of the inorganic powder, or bound by ionic bonding or condensation reaction.

12. The cosmetic composition according to claim 1, wherein the cosmetic composition is used in a cosmetic composition for skin care or a cosmetic composition for make-up.

13. A cosmetic process for care of and/or makeup of a skin of a subject, comprising applying a cosmetic composition according to claim 1 to the skin of a subject.
